(19) United States
(12) Patent Application Publication
(10) Pub. No.: US 2016/0143654 A1
(43) Pub. Date: May 26, 2016

(54) Skin Tag Removal Device

(71) Applicant: Melissa Zantop, Denver, CO (US)

(72) Inventor: Melissa Zantop, Denver, CO (US)

(21) Appl. No.: 14/934,980

(22) Filed: Nov. 6, 2015

Related U.S. Application Data
(60) Provisional application No. 62/082,767, filed on Nov. 21, 2014.

(51) Int. Cl. A61B 17/32; A61B 17/22

(52) U.S. Cl. CPC A61B 17/32; A61B 17/22031

(57) ABSTRACT

A skin tag removal device for removing skin tags from a user's body. The skin tag removal device includes a clamp having a first arm and a second arm pivotally affixed to one another by a resistance element. The resistance element helps to bias the arms in a closed configuration. In operation, the user can squeeze the second end of the arms which serve as handles in order to separate the first ends of the arms. The first ends of the arms include projections thereon that are in contact with one another when the clamp is in the closed configuration. The clamp can be secured to the stalk of a skin tag wherein the projections contact opposing sides of the base of the skin tag in order to cut off blood supply to the skin tag, causing the skin tag to fall off.

### CROSS REFERENCE TO RELATED APPLICATION

This application claims the benefit of U.S. Provisional Application No. 62/082,767 filed on Nov. 21, 2014. The above identified patent application is herein incorporated by reference in its entirety to provide continuity of disclosure.

### BACKGROUND OF THE INVENTION

### Field of the Invention

The present invention relates to skin tag removal devices. More specifically, the present invention provides a clamp for attachment to a skin tag in order to facilitate removal of the same.

Skin tags are small flaps of tissue that grow on a person's skin. Skin tags are commonly formed on the neck, chest, back, and armpits. Skin tags are generally not harmful, however, skin tags can become irritated by contact and can be unsightly developments. Skin tags are typically treated by cutting the skin tag from the skin using scissors or a scalpel. However, this process can be painful and can result in irritation or infection if not performed properly. Alternately, the skin tag can be frozen using liquid nitrogen, which can also be painful and can irritate the surrounding skin.

Devices have been disclosed in the prior art that relate to skin tag removal devices. These include devices that have been patented and published in patent application publications. These devices generally relate to adhesive members that can be placed over the skin tag to facilitate removal thereof, such as U.S. Pat. No. 8,128,637, U.S. Published Patent Application Number 2013/0123806, U.S. Published Patent Application Number 2012/0330333, U.S. Pat. No. 6,375,652, and U.S. Published Patent Application Number 2008/0319475.

These prior art devices have several known drawbacks. The devices in the prior art provide adhesive members for helping to remove skin tags, however, these devices do not provide clamps that can be secured to a portion of the skin tag in order to cut off the flow of blood to the skin tag, causing the skin tag to fall off. The clamp device can be reused on additional skin tags as necessary.

In light of the devices disclosed in the prior art, it is submitted that the present invention substantially diverges in design elements from the prior art and consequently it is clear that there is a need in the art for an improvement to existing skin tag removal devices. In this regard the instant invention substantially fulfills these needs.

### SUMMARY OF THE INVENTION

In view of the foregoing disadvantages inherent in the known types of skin tag removal devices now present in the prior art, the present invention provides a new skin tag removal device wherein the same can be utilized for providing convenience for the user when facilitating removal of a skin tag from a user's body.

The skin tag removal device comprises a clamp having a first arm pivotally affixed to a second arm by a resistance element. The resistance element may include a spring or a flexible member. The clamp can be moved between an open and closed configuration, wherein the first end of the arms is in contact in the closed configuration. The first ends of the arms further include projections thereon that are in contact in the closed configuration and that are adapted to be secured to the base of a skin tag in order to cut off the flow of blood into the skin tag, causing the skin tag to fall off.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a perspective view of the skin tag removal device in a closed configuration.

FIG. 1B shows a perspective view of the skin tag removal device in an open configuration.

FIG. 2A shows a planar view of the top of the skin tag removal device.

FIG. 2B shows a planar view of the bottom of the skin tag removal device.

### DETAILED DESCRIPTION OF THE INVENTION

Reference is made herein to the attached drawings. Like reference numerals are used throughout the drawings to depict like or similar elements of the skin tag removal device. For the purposes of presenting a brief and clear description of the present invention, the preferred embodiment will be discussed as used for removing skin tags from a user's body. The figures are intended for representative purposes only and should not be considered to be limiting in any respect.

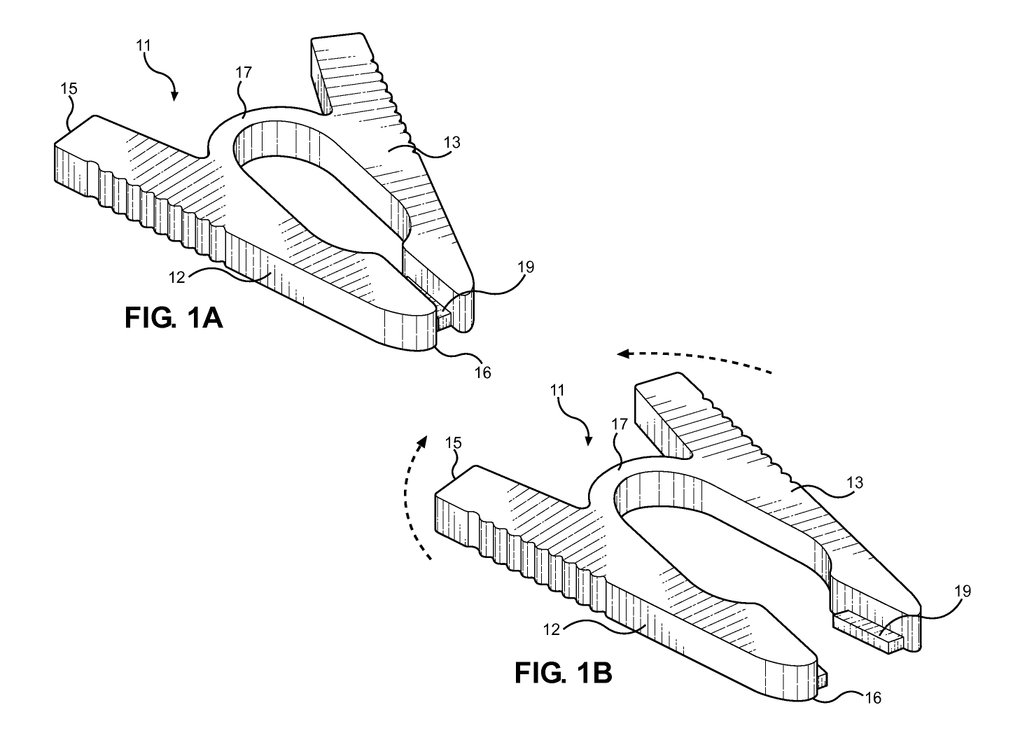

Referring now to FIGS. 1A and 1B, there are shown views of the skin tag removal device in an open and closed configuration, respectively. The skin tag removal device 11 comprises a clamp having a first arm 12 pivotally secured to a second arm 13 via a resistance element 17. The first arm 12 and second arm 13 each include a first end 16 opposite a second end 15. Preferably, the resistance element 17 is affixed to a central or middle portion of the first arm 12 and second arm 13 and extends between the first and second arms 12, 13 The resistance element 17 may include a spring-biased hinge in some embodiments. In alternate embodiments, the resistance element 17 includes a flexible plastic member.

The clamp is movable between an open configuration and a closed configuration, wherein the resistance element 17 biases the clamp in the closed configuration. The second ends 15 of the first arm 12 and second arm 13 serve as handles for the user to hold and manipulate. By pressing or squeezing the second ends 15 of the arms 12, 13 towards one another, the first ends 16 of the arms 12, 13 are separated. When the user ceases to apply pressure to the second ends 15 of the arms 12, 13, the clamp returns to a closed configuration in which the first ends 16 of the arms 12, 13 are adjacent to one another.

The first end 16 of each arm 12, 13 is tapered so as to provide a smaller cross sectional area than the remainder of the arm 12, 13. This allows for greater visibility when the user is attempting to place the clamp on the stalk or base of the skin tag. The first ends 16 further comprise a projection 19 thereon. The projections 19 extend inward from the arms 12, 13 and the projections 19 contact one another when the clamp is in the closed configuration. The arms 12, 13 are separated by a space so that the arms 12, 13 do not contact one another along the length thereof, and the arms 12, 13 are only in contact at the first end 15 thereof. The projections 19 extend from the lower end, or bottom, of the first ends 16 in order to allow the projections to grasp the base of a skin tag when in use. The projections 19 are preferably rectangular in configuration.

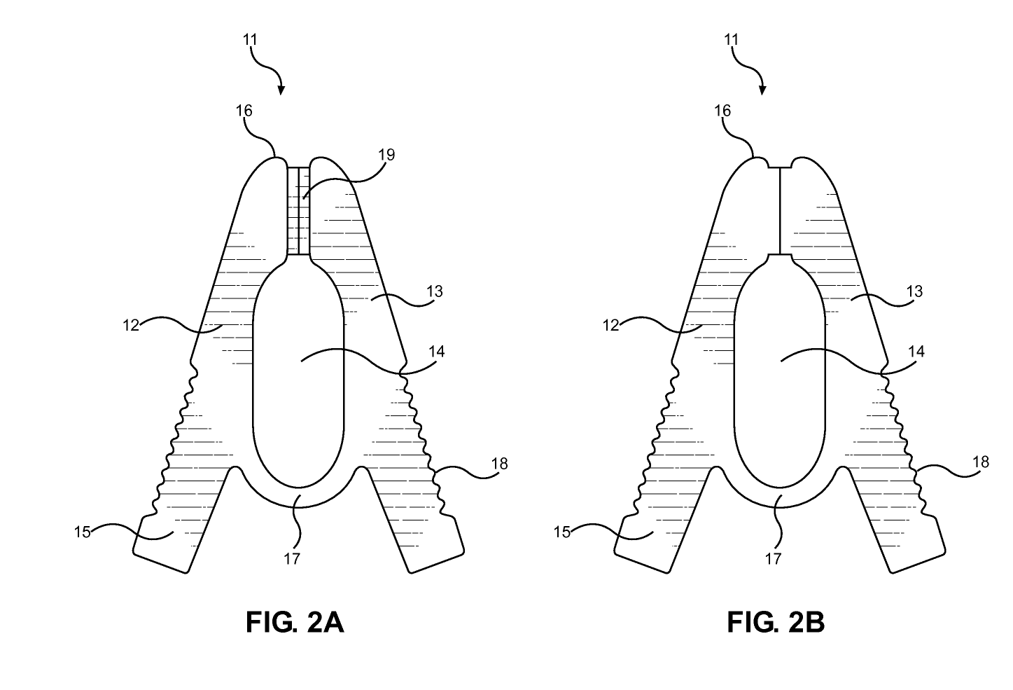

Referring now to FIGS. 2A and 2B, there are shown planar views of the top and bottom of the skin tag removal device. In operation, the user can hold the second ends 15 of the arms 12, 13 and squeeze the second ends 15 towards one another so as to separate the first ends 16 of the arms 12, 13. The arms 12, 13 preferably include ridges 18 thereon so as to improve the user's grip on the device. The user can then position the clamp adjacent to a skin tag on the user's body so that the skin tag is located between the projections 19 on the first end 16 of the clamp. The second end 15 of the arms 12, 13 are then released, so that the clamp returns to a closed configuration. The projections 19 press against opposing sides of the base of the skin tag. The force or pressure provided by the resistance element 17 secures the clamp in position on the skin tag and also helps to restrict or eliminate blood flow to the skin tag. The clamp is maintained on the skin tag for a period of time until the skin tag eventually falls off of the user's body. In this way, the user can easily remove skin tags without the use of scissors, scalpels, or chemicals. Further, the skin tag removal device 11 can then be reused to treat additional skin tags as desired.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A skin tag removal device, comprising:
a clamp having a first arm pivotally affixed to a second arm via a resistance element, wherein the clamp is movable between an open configuration and a closed configuration;
wherein the first arm and the second arm each include a first end and a second end;
wherein the first end of the first arm and the first end of the second arm each include a projection thereon, wherein the projections contact one another when the clamp is in the closed configuration.

2) The skin tag removal device of claim 1, wherein the resistance element is a spring-biased hinge.

3) The skin tag removal device of claim 1, wherein the resistance element is a flexible plastic member.

4) The skin tag removal device of claim 1, wherein the second end of the first arm and the second end of the second arm include ridges thereon.

5) The skin tag removal device of claim 1, wherein the first end of the first arm and the first end of the second arm are tapered.

6) The skin tag removal device of claim 1, wherein the projection on the first arm and the projection on the second arm are substantially rectangular in configuration.

7) The skin tag removal device of claim 1, wherein the clamp is biased in a closed configuration.

8) The skin tag removal device of claim 1, wherein the projection on the first arm and the project on the second arm are located on the lower end of the first arm and the second arm, respectively.
